(19) United States
(12) Patent Application Publication
(10) Pub. No.: US 2001/0032938 A1
(43) Pub. Date: Oct. 25, 2001

(54) Through-the-lens-collection of secondary particles for a focused ion beam system

(76) Inventors: Robert L. Gerlach, Portland, OR (US); Mark W. Utlaut, Scappoose, OR (US)

Correspondence Address: MICHAEL O. SCHEINBERG, P.O. BOX 164140, AUSTIN, TX, 78716-4140, US

(21) Appl. No.: 09/780,876

(22) Filed: Feb. 8, 2001

Related U.S. Application Data
(60) Provisional application No. 60/181,248, filed on Feb. 9, 2000.
(60) Provisional application No. 60/205,605, filed on May 18, 2000.

(51) Int. Cl. G21G 5/00; A61N 5/00

(52) U.S. Cl. 250/492.3

(57) ABSTRACT

Secondary particles are collected along the optical axis of a focused ion beam system, thereby eliminating the need for side-mounted detectors that make close packing of multiple columns difficult and that require the final lens to be spaced further away from the target. Secondary particles can be collected through the lens and then diverted away from the optical axis of the ion column for detection without significantly degrading the resolution of the focused ion beam. Secondary particles can also be collected using a conductive plate connected to an amplifier to detect the electrical current caused by the secondary particles.

### RELATED APPLICATIONS

This application claims priority from U.S. Provisional Patent Application No. 60/181,248 filed Feb. 9, 2000 and from U.S. Provisional Patent Application No. 60/205,605 file May 18, 2000, both of which are hereby incorporated by reference.

### TECHNICAL FIELD OF THE INVENTION

The present invention relates to the field of focused ion beam (FIB) systems, and in particular, to the collection of secondary particles in FIB systems.

### BACKGROUND OF THE INVENTION

Thin film head trimming and other nanofabrication applications suffer from throughput limitations, that is, the focused ion beam systems are unable to process products as quickly as desired. One approach to increasing processing speed is to increase the current in a given beam, thereby increasing the rate at which material is removed or deposited. Unfortunately, systems are approaching fundamental limits for beam current within the small beam diameters required.

Another solution to increasing throughput is simply to use additional FIB systems. Because FIB systems are complex and include a variety of subsystems, each individual system is costly.

To increase throughput in electron beam lithography and semiconductor inspection areas, researchers have begun using multiple electron field emitters or photoemission sources in a single system. For example, multiple electron beam systems are described in U.S. Pat. No. 4,390,789 to Smith for “Electron Beam Array Lithography System Employing Multiple Parallel Array Optics Channels and Method of Operation” and U.S. Pat. No. 5,981,962 to Groves et al. for a “Distributed Direct Write Lithography System Using Multiple Variable Shaped Electron Beams.”

These multi-beam electron beam system designs do not readily transfer to ion beam systems because the ion optical columns operate at much higher voltages and therefore present design problems that are not present in electron beam systems. Designing an array of high voltage FIB columns that operate at beam voltages of about +30,000 V presents problems that are quite different from those involved in designing an array of electron beam columns, which typically operate between about −500 to −5000 V, for lithography or inspection. Moreover, the higher voltage power supplies are bulkier and more costly than low voltage ones required for electron beam systems, making it difficult to produce a compact and competitively priced multicolumn FIB system.

U.S. Pat. No. 5,945,677 to Leung et al. for a “Focused Ion Beam System” describes two approaches for multiple ion beam systems using one or more plasma ion sources. In the first approach, a single multicusp plasma ion source is used to produce multiple beamlets. The second approach employs multiple FIB units, each having a separate ion source and acceleration column. Such systems are complex, and no large area source, multiple column systems are currently commercially available. Furthermore, multicusp sources, which are about 100 times less bright than liquid metal field ion sources, have so far not achieved sufficient brightness to be commercially practical.

Thus, a practical, cost effective solution for increasing focused ion beam throughput while maintaining resolution is still needed.

### SUMMARY OF THE INVENTION

An object of the present invention is to provide for the collection of secondary charged particles in focused ion beam systems in a manner that allows for efficient collection and a more compact system design.

The present invention includes a method of increasing the throughput of a FIB system, a FIB system capable of increased throughput, and a method of making and using the FIB system. The invention also includes several novel aspects of the FIB system, including the modular design of the gun chambers, the design of the electrodes, including their electrical isolation, the secondary particle collection system, and the electrode voltage application scheme. Although this disclosure describes a FIB system having multiple ion optical columns, many of the innovation described herein, such as the secondary particle collection techniques, are also applicable to systems having a single ion optical column.

A preferred FIB system of the present invention comprises multiple ion guns, each preferably including a Liquid Metal Ion Source (LMIS) and associated with a corresponding FIB optical column. The beams from the multiple columns are directed to one or more targets in a primary vacuum chamber. The multiple guns increase the number of ions impacting the target or targets and therefore increase the processing rate. For example, the multiple beams can operate on different wafers, or on different parts of a single wafer, with the wafer or wafers being in a single primary vacuum chamber. Because each of the multiple columns has substantially the same resolution and beam current as that of a column in a single gun FIB system, accuracy and precision is not degraded as processing speed is increased. Because the multiple columns share a primary vacuum chamber and can share other facilities, such as power supplies, a computer and a user interface, the initial cost and operating cost of a system of the invention is greatly reduced compared to the costs associated with multiple, separate complete FIB systems.

Preferably, each FIB gun is placed in a vacuum chamber, referred to as a gun chamber, that can be evacuated independently from the primary vacuum chamber. Multiple such gun chambers, each containing one or more FIB guns, can be placed in parallel to form a large array of guns for operating on one or more targets in the primary vacuum chamber. A common, ganged vacuum valve for each gun chamber can isolate the gun chamber from the main chamber. Moreover, the gun chambers can be evacuated and sealed prior to installation, thereby avoiding the loss of production that would occur if the gun chamber were evacuated after installation. By separating the gun chamber or chambers from the main chamber, gun chambers can be replaced without disrupting the vacuum in the main chamber, thereby avoiding the introduction of contaminants and saving the time required to re-evacuate the main chamber. By having guns in separate gun chambers, some guns can be replaced without disturbing others. A gun chamber can be replaced as a module in the field, with the repairs or replacements of individual guns being performed at the factory.

Each gun has a corresponding ion optical column, with some of the column elements preferably being placed below the guns in the main system chamber to form an array of columns. To reduce complexity and to increase the placement precision between columns, column elements can be made common between the columns by using bars with precision cut holes to form the lens elements.

The present invention also includes systems for collecting secondary particles. As opposed to convention side-mounted detectors, secondary particles are collected along the optical axis of the ion beam column. The secondary particles are then either deflected off the ion beam column axis to a detector or the secondary particles are detected by a detector positioned along the axis and having a hole for passing the ion beam. The on-axis detector may be positioned on either above or below the final lens.

In one preferred embodiment, the secondary particles are accelerated through the final lens in a manner such that the resolution of the ion beam is not significantly degraded and then the secondary particles are deflected away from the ion column optical axis toward a detector. This through-the-lens detector arrangement allows the sample to be placed close to the final lens, thus shortening its focal length and providing improved column optical performance (greater current into the same beam diameter). The system for collecting secondary particles through the final ion lens can be used on multi-column or single column ion beam systems. In another preferred embodiment, a conductive plate is used to detect the secondary particles.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 is a side, cross-sectional view of a single FIB column set containing five FIB columns.

FIG. 2A is a top view of three column sets fastened together to form a 15 column array.

FIG. 2B is a side view of these three column sets.

FIG. 3 is an end cross sectional view of a gun set using high voltage insulators.

FIG. 4 is a cross-sectional view of a FIB column using a through-the-lens secondary particle detector.

FIG. 5 is a cross-sectional view of another FIB column using a through-the-lens secondary particle detector.

FIG. 6 is an electron optics computer simulation of the secondary electron trajectories from the sample through the lenses of FIG. 4.

### DETAILED DESCRIPTION OF THE PREFERRED EMBODIMENTS

The embodiments described below accomplish several difficult design goals for multi-column FIB systems. The optical elements are sufficiently electrically isolated to maintain the required high operating voltages. In some embodiments, the number of high voltage power supplies is reduced from the number that would be required in multiple independent columns. The voltage level of the high voltage power supplies are also reduced from that of conventional FIB systems. In addition, the difficulties of keeping multiple LMIS's (Liquid Metal Ion Sources) operating and maintained with minimum down time is addressed by using a vacuum sealable, multiple gun chamber as described below. Many of the innovation described herein, such as the secondary particle collection techniques, are also applicable to systems having a single ion optical column.

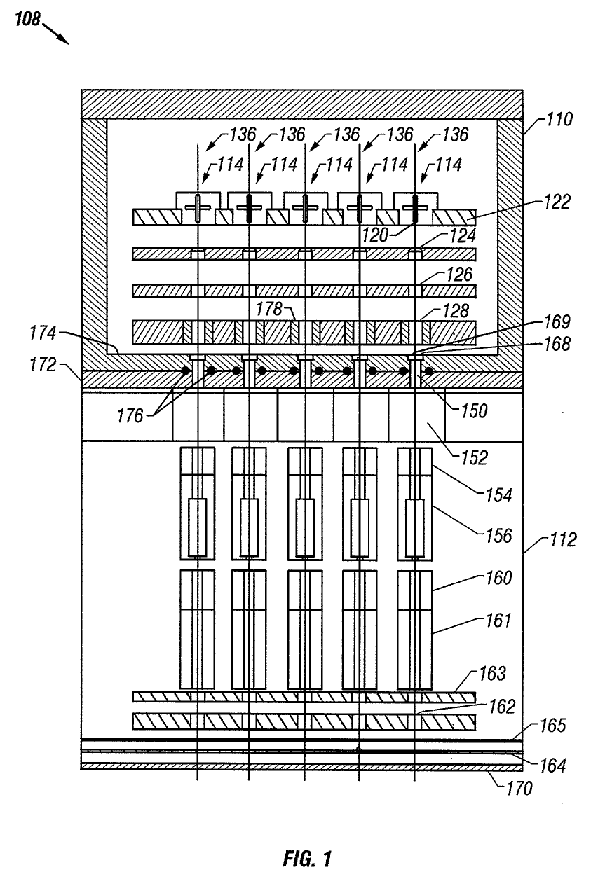

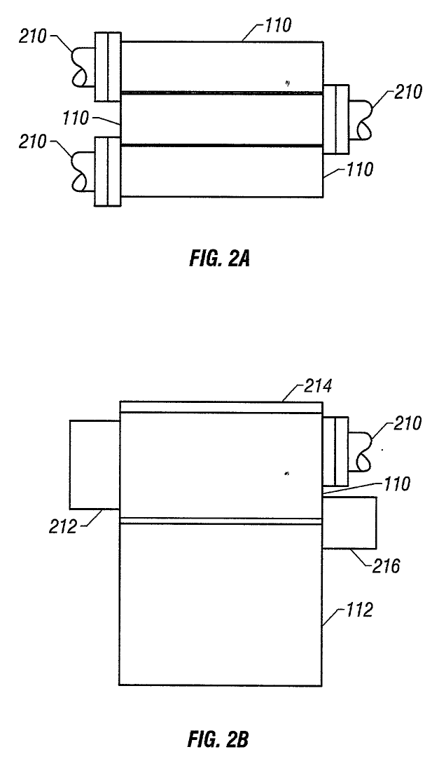

FIGS. 1, 2A, and 2B show a multi-column FIB array using LMIS's. FIG. 1 shows a multi-column FIB system 108 that includes a gun vacuum chamber 110 and a primary vacuum chamber 112. Gun chamber 110 is a single, sealable vacuum chamber that includes a set of ion guns 114. Gun chamber 110 can be replaced as a unit and has its own vacuum pump, preferably an ion pump (not shown). When one of the guns 114 in gun chamber 110 fails, the entire gun chamber 110 can be replaced with another gun chamber 110 that is already evacuated to an ultra high vacuum and ready to begin operation. Thus, multi-column system 108 does not need to remain out of production while the gun chamber is being evacuated.

Each ion gun 114 includes an emitter 120, a suppressor 122, an extractor 124, an acceleration lens 126, a deceleration lens 128 and a ground element 169. The four elements 124, 126, 128 and 169 of each column together are referred as the “lens 1” of the column. Although FIG. 1 shows a lens 1 comprising four lens elements, other lens designs can be used for lens 1. Also, some or all of the elements of lens 1 could alternatively be positioned in primary vacuum chamber 112.

Each ion gun 114 forms part of an ion optical column 136 that also includes an aperture 152, a steering element 154, a blanking element and Faraday cup 156, dual deflection elements 160 and 161, second lens elements 163, 162 and 165 (referred to collectively as the “lens 2”), and a detector 164. At the bottom of each column is a work piece or target 170, such as a semiconductor wafer. An isolation valve 150 at the bottom of each gun 114 selectively closes a beam hole 168, thereby vacuum isolating gun chamber 110. The isolation valves 150 of the column in a gun chamber 110 are preferably “ganged,” that is, connected in a manner so that all valves are opened or closed together. The detectors 164 for the columns 136 are constructed so that each column's secondary electrons can be independently detected. The aperture 152 preferably comprises an automatic variable aperture. Such apertures are known and details are not shown in FIG. 1. A gas injection system can optionally be used with apparatus of FIG. 1 to inject a gas for ion beam assisted deposition or for enhanced etching. The construction and operation of such systems are known and are described, for example, in U.S. Pat. No. 5,435,850 to Rasmussen.

The gun elements, that is, emitters 120, suppressors 122, extractors 124, acceleration lenses 126, deceleration lenses 128, and ground element 169 are preferably contained in gun chamber 110. The number of guns in gun chamber 110 is preferably limited to about five. If one of the emitters 120 fails, then the exchange of a five-gun set is easier and less costly than replacing a larger number of guns, such as ten or more guns. Moreover, the restarting of five emitters in parallel is also much less prone to failure than restarting a larger number simultaneously. The set of ganged isolation valves 150 for the set of guns simultaneously isolates the beam holes 168 in the ion beam paths at the bottom of gun chamber 110 from the primary vacuum chamber 112.

Valves 150 are preferably formed by a bar 172 that moves relative to bottom portion 174 of gun chamber 110. When valves 150 are open, the openings in bar 172 line up with the openings 169 in bottom portion 174. To seal gun chamber 110, bar 172 is shifted so that the holes in bar 172 are offset from the holes in bottom portion 174, and O-rings 176 form a seal between a solid portion of bar 172 and bottom portion 174. Before shifting bar 172, it is preferably lowered away from O-rings 176 to prevent damage to the O-rings that can create contamination and vacuum leakage. After bar 172 is shifted, it is raised again into contact with O-rings 176 to create a vacuum seal to isolate gun chamber 110. Primary chamber 112 can be exposed to air when gun chamber 110 is removed or alternatively, primary chamber 112 can be made sealable by using a second set of valves (not shown). Details of the mounting of gun chamber 110 to primary chamber 112 are conventional and not shown.

A multiple ion column system could use a single gun chamber 110 or multiple gun chambers. FIG. 2A shows a top view of an arrangement of multiple linear gun chambers 110 grouped to form a two-dimensional array of fifteen guns. The number of guns per gun chamber can be varied, as well as the number of gun chambers to produce a system having the desired number of FIB columns for a particular application. FIG. 2A shows an outlet 210 from each gun chamber 110 to an associated ion pump.

FIG. 2B is a side view of the multiple gun chamber system of FIG. 2A. FIG. 2B shows also a location for high voltage feed-throughs 212, a flange 214 at the top of a gun chamber 110, and an actuator 216 for ganged gate valves 150.

The construction of the optical elements, such as extractors 124, acceleration lenses 126, and deceleration lenses 128 in gun chamber 110, can be simplified by using flat bars with holes to form lens elements. This construction technique can also be used to construct optical elements in the primary vacuum chamber. Using a single bar to form corresponding lens elements in different columns with a gun chamber can reduce the number of high voltage power supplies required.

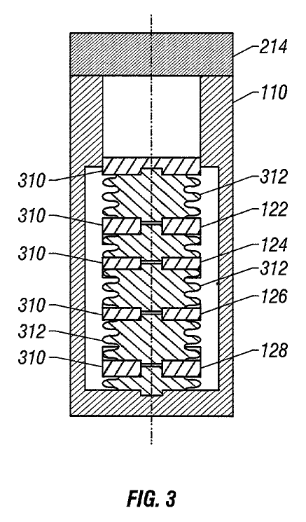

FIG. 3 is a cross-sectional view of a gun chamber 110 showing bars 310 used to form optical elements. Bars 310 form suppressors 122, extractors 124, acceleration lenses 126, and deceleration lenses 128. Bars 310 are electrically isolated from each other and from the chamber itself using HV (High Voltage) insulator disks 312 composed preferably of a ceramic material. Other means, such as dielectric balls, can be used to isolate the gun HV elements. The assembly may be glued together using a suitable epoxy or other means known in the art. After the bars and insulators are installed in the casing of gun chamber 110, the assembly can optionally be machined to provide additional accuracy in shaping and aligning the lens elements.

The optical elements can be formed directly by the holes in a conductive bar, as shown with regard to acceleration lenses 126. A common voltage is thus applied to all lenses formed by the bar, reducing the number of high voltage power supplies required for the multi-column system. The number of high voltage power supplies can be further reduced by using a common high voltage supply for corresponding bars in multiple gun chambers. Conductive bars are typically made of a titanium alloy having a low thermal coefficient of expansion.

Optical elements can also be formed by inserts placed into holes in a bar. For example, FIG. 1 shows the use of lens inserts 178 in the bar 310 forming deceleration lenses 128. The bar in which lenses 128 are formed is constructed from an insulating material, for example, a ceramic material such as alumina, and the lens inserts are composed of a conductive material, preferably a titanium alloy which has is low thermal coefficient of expansion that is similar to that of the alumina bar. The alumina bar provides high voltage isolation to the individual lenses 128 with respect to the bars 310. Voltage is applied to the individual lenses by wires connected to the lenses in a conventional manner, such as conductive silver epoxy or using connector pins. Alternatively, metal films can be placed upon the insulator bar to replace the wires.

Another method of providing high voltage insulation to lenses 128 entails using a conductive bar 310, with an insulating insert placed in a hole in the bar, and then a conductive lens placed in the insulating insert. Such inserts can be glued into insulator material, which can then be glued into the bars. Lenses formed by inserts can also be post machined, that is, machined after assembly, for additional accuracy in shape and alignment. These construction methods that individually isolate lenses can be particularly useful for suppressor lenses 122, extractor lenses 124, or individual elements of lens 1 or lens 2.

FIG. 1 shows inserts used only on the deceleration lens 128. Deceleration lens 128 can be operated near ground potential, which simplifies the power supply requirements for lens 1. Isolating lens elements allows the voltages in individual columns to be controlled. For example, the voltage on one of extractor lenses 124 can be individually boosted about 2 kV above the common extractor voltage to start or restart the individual emitter in the corresponding column. The extractor lens 124 can then return to or near the common extractor voltage for normal operation. Optical elements that are isolated can still use the common high voltage supply, but isolated elements can also be floated at a voltage above or below the common voltage, thereby reducing the number of high voltage power supplies required.

Charged particle signal detection capability for imaging can be accomplished by a traditional side mounted electron multiplier or scintillator means, or by other novel methods described below. Traditional side-mounted secondary particle collection systems require that the final lens be placed at a sufficient distance from the sample to accommodate the particle collection apparatus. Increasing the distance from final lens to the sample necessitates using a final lens having a longer focal length, which typically degrades the resolution of the FIB system. In the prior art, channel plate detectors have been positioned along the ion column optical axis between the final lens and the sample, but channel plates at this location increase the lens focal length, suffer reliability problems and do not have a long life.

In the embodiments described below, secondary particles are collected along the optical axis of the ion beam column. The secondary particles are then either deflected away from the ion beam column optical axis to a detector or the secondary particles are detected by a detector positioned along the optical axis and having a hole for passing the ion beam. The detector may be positioned on either side of the final lens.

For many nanofabrication applications, the beam current can be greater than a nanoampere. At this level of current, an amplifier or amplifiers can be attached directly to detector plate 164 below lens 2 to detect a current caused by secondary charged particles. As described above, detectors 164 for the columns 136 are constructed so that each column's secondary electrons are independently detected. For example, the individual detectors could be electrically isolated each other, for example, by using inserts as described above, or secondary particle collection could be performed using only one column at a time during imaging. Separate detector plates for each column could also be used. Detector 164 can be electrically biased to collect positive or negative charged particles. Skilled persons can readily determine an appropriate bias by adjusting the bias to provide an adequate electrical current for detection without significantly degrading the resolution of the ion beam.

Alternatively, secondary particles, electrons or positive or negative ions, can be collected through the final lens, referred to as through-the-lens (TTL) collection. After the secondary particles travel back through the final lens, they can be detected by an on-axis collection system, such as one similar to detectors 164 (FIG. 1), a channel plate, or a scintillation detector, or by a preferred off-axis detection system as described below.

Through-the-lens electron detection in an electron beam column is known and described, for example, in PCT Publication WO 99/34397 of Krans et al. In the Krans et al. design, the lens 2 center element and upper element (and optionally the lower element) are biased to positive potentials to draw the electrons from the sample up and above the lens, where they are detected by a channel plate electron multiplier, which is oriented roughly perpendicularly to the ion column axis and which has a hole in its center to pass the primary beam.

It is preferable to use a TTL detection system in focused ion beam systems to provide a short focal length final lens. However, the optics in a FIB column is significantly different from a low voltage SEM column, such as the Krans et al. design. A typical FIB column operates at about 30 kV beam voltages and the final lens is an einzel lens—the center element is at a high positive potential and the upper and lower elements are at ground potential. Thus significant modification to the ion optics, different from those described by Krans et al. for an electron column, are required to provide TTL capability in an ion column.

A key realization is that the mass-to-charge ratio for ions employed in FIB systems is orders of magnitude higher than for electrons, and in addition, the energies are much higher. Thus a simple magnetic field transverse to the primary and secondary electron beam paths can be employed to deflect the electrons off-axis with minimal disturbance to the primary ion beam. As an example, applicants used gallium ions with 30 kV beam voltage and electrons extracted upwards from the sample through the final lens with voltages of 2000 V.

The strength of the bending magnet is given by the cyclotron radius equation

R=mv/Be (1)

where m is the mass, v is the velocity of the charged particle, B is the magnetic field strength, and e is the electron charge. Substituting the energy and mass into Equation 1 gives

R=(2Em)^{½}/Be (2)

For electrons, it can be shown that B R(gauss-cm)=3.37 E^{½} (where E is in eV). Therefore, if we use R=5 mm and E=2000 V for the secondary electrons accelerated upward beyond the final lens, then B=301.4 gauss.

This bending magnet is designed so as not to disturb the primary ion beam very much. Fortunately, gallium primary ions have the same charge as electrons but each gallium ion has a mass about 160,000 times the mass of an electron. Furthermore, the electron energies are typically about 15 times less than the ion energy. Using Equation 2, we see that the cyclotron radius for the primary ions is typically about 1550 times larger than the electron cyclotron radius or about 7.75 meters. Hence the disturbance to the primary ion beam path is small. In fact the path deviation is only about 1.2 milli-radians, which can be very easily corrected with beam steering. It can also be shown that the predominate aberration introduced into the primary ion beam by the magnetic field is chromatic and can mostly be neglected.

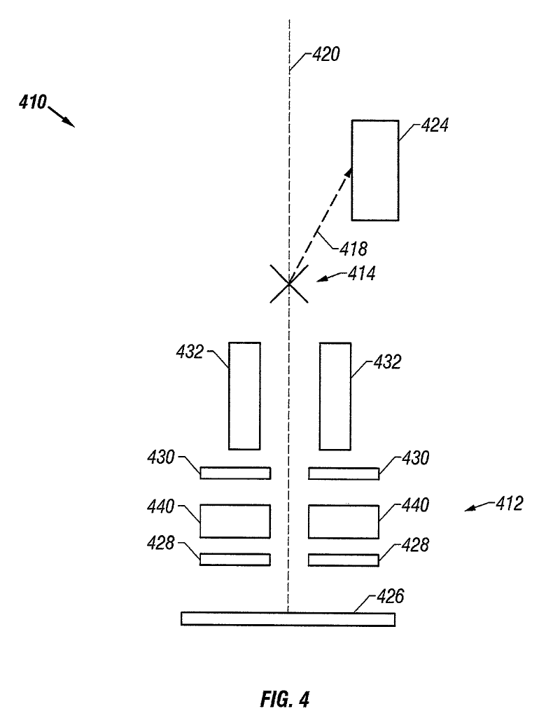

FIG. 4 shows such a TTL detection system for an ion column 410 in which low energy secondary electrons from the sample, having energies of about 5 eV (electron volts), are accelerated up through the lens 412 by positive potentials on the elements of lens 412, the deflector plates 432 and magnetic deflector 414. The TTL system in FIG. 4 utilizes a magnetic deflector 414 to deflect the secondary electrons 418 off to the side while allowing the high mass-to-charge ratio primary ions 420 to pass nearly straight through column 410. Alternatively, a Wein filter or an electrostatic deflection device could be used. An electron detector 424, such as a scintillator, continuous dynode multiplier, or channel plate, is then placed to the side for collecting and amplifying the electron signal for processing by standard FIB video electronics.

In the embodiment of FIG. 4, a sample 426 and a lower lens element 428 are maintained at approximately ground potential. An upper lens element 430 is biased to between about +500 and +5000 volts with respect to ground to continue the secondary electron velocities upward beyond the lens 412. Similarly, electrostatic deflector plates 432 and deflector 414 are biased to between about +500 and +5000 volts to continue this upward velocity of secondary electrons 418 towards electron detector 424, the input of which are similarly biased.

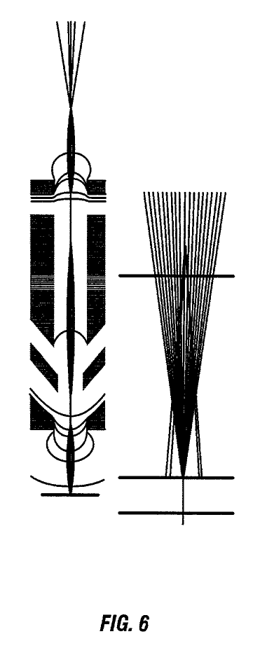

FIG. 6 is an electron optics computer simulation of the secondary electrons traveling from the sample back through the lens shown in FIG. 4. The approximately 5 eV secondary electrons are accelerated rapidly by the lens element 440, which is at high positive potential, such as about 20,000 Volts. These electrons are decelerated as they pass through the lens element 430 and the deflection electrodes 432, but the secondary electrons still maintain trajectories that remain relatively close to the column axis. Magnetic deflector 414 or other separation device then directs the electrons toward the detector 424.

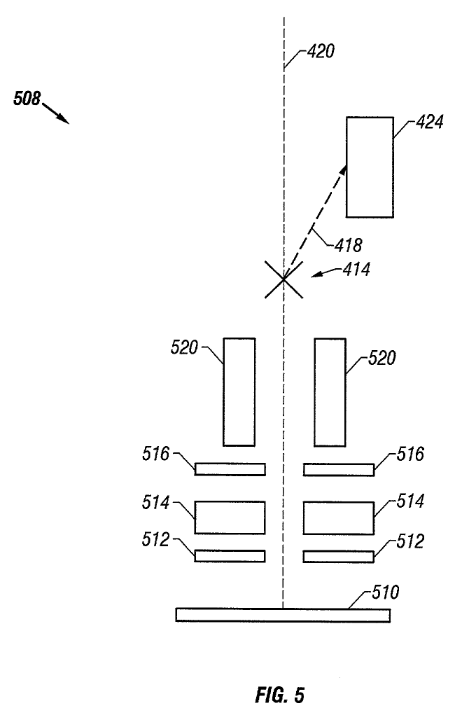

FIG. 5 shows an alternate ion column 508 design using a TTL secondary electron detector. A sample 510 and a lower final lens element 512 are each biased about −2000 V negative to propel the electrons back through the lens. If it is desired to collect secondary positive ions instead of electrons, sample 510 and lower final lens element 512 can be biased to about +2000 V. Center lens element 514 is biased to approximately +20,000 V. Lens element 516, electrostatic deflector elements 520 and deflector 414 need not be positively biased, which simplifies the electronics and the optics construction. If the ion beam systems include other devices, such as gas injectors, these devices are also biased to the same potential as the sample.

The apparatus in FIG. 4 also may be used to detect secondary positive ions from the sample. To collect secondary positive ions, the lens 2 element 440 is biased to a negative potential. (Lens 2 is then an acceleration lens). In addition, electrostatic deflector 432, the deflector 414 and the input of particle detector 424 are negatively biased. Similarly, the potentials in FIG. 5 may be changed to collect and detect positive secondary ions. A quadrupole or other mass spectrometer can also be placed in the position of detector 424 to perform Secondary Ion Mass Spectrometry. The appropriate biasing of the column and detector may be employed to detect either positive or negative ions.

For thin film head trimming or other applications, the ion beams are typically tilted about +/−3 degrees with respect to the normal to the sample surface. This beam tilting is to achieve undercutting or to yield cuts to the sides of the head with walls more normal to the head surface. This +/−3 degree tilt can be achieved, for example, by tilting every other row of columns by about +/−3 degrees with respect to the normal to the sample surface.

The inventions described above can be embodied in a variety of systems, and the advantages delineated below can be provided in many or all of the embodiments. Because the embodiments will vary with the goals of a particular application, not all advantages will be provided, or need to be provided, in all embodiments.

An advantage of the invention is an increase in the processing speed by providing a system including multiple ion guns capable of operating simultaneously on one or more targets.

Another advantage of the invention is that it provides a system in which the multiple ion guns operate on one or more targets in a single primary vacuum chamber.

Another advantage of the invention is that it provides a system in which the multiple ion guns are in a gun chamber capable of being vacuum isolated from the main chamber, that is, the gun chamber is capable of being evacuated or exposed to atmosphere independently, without disrupting the vacuum in the main chamber.

Another advantage of the invention is that it provides a system in which the multiple ion guns are positioned in multiple gun chambers, each gun chamber containing one or more ion guns and each gun chamber capable of being vacuum isolated from the main chamber and from each other.

Another advantage of the invention is that it provides a multiple ion gun system in which a portion of the ion column elements are in the primary vacuum chamber.

Another advantage of the invention is that it provides a system in which an ion gun or set of ion guns in one chamber can be replaced while maintaining a vacuum in the main chamber and in other gun chambers.

Another advantage of the invention is that it provides a system that uses multiple ion guns and provides the capability to detect secondary particles emitted from a sample at the target point of each of the multiple guns.

Another advantage of the invention is that it provides charged particle optical elements in parallel for multiple columns and a method of efficiently manufacturing such elements.

Another advantage of the invention is that it provides such charged particle optical elements with at least one of the optical elements being individually controllable.

Another advantage of the invention is that it provides an electrode design for a multiple column focused ion beam system that reduces the number of high voltage power supplies required for the system.

Another advantage of the invention is that it provides a multiple column focused ion beam system using fewer high voltage power supplies than the number of columns.

Another advantage of the invention is that it provides an electrode design and voltage application scheme that reduces the voltage requirement of the high voltage power supply.

Another advantage of the invention is that it reduces the cost of processing multiple targets simultaneously from the cost of using multiple, single beam focused ion beam systems.

Another advantage of the invention is that individual emitters can be restarted by individually increasing the extraction voltage of that particular gun and not disturb the other gun voltages. This can be achieved either by increasing the extractor voltage with respect to the emitter/suppressor elements by using isolated extractor elements, or by increasing the emitter/suppressor voltage with respect to the common extractor voltage for that particular gun.

Although the present invention and its advantages have been described in detail, it should be understood that various changes, substitutions and alterations can be made to the embodiments described herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

We claim as follows:

1. A focused ion beam apparatus including a detector for detecting secondary particles, comprising:
an ion gun for generating primary ions and directing them toward a target;
a lens for focusing the primary ions onto the target and for passing secondary particles emitted from the target and traveling in a direction opposite to that of the primary ions; and
a detector for detecting the secondary particles emitted from the target and passing through the lens.

2. The apparatus of claim 1 in which at least a portion of the lens is biased to a positive electrical potential relative to the target to accelerate negatively charged secondary particles toward the lens and away from the target.

3. The apparatus of claim 1 in which:
the primary ions travel along a primary ion path; and
the detector is positioned off the primary ion path and further comprising
a secondary particle deflector for deflecting away from the primary ion path secondary particles that travel through the lens and toward the detector, the secondary particle deflector allowing the primary ion beam to pass toward the target.

4. The apparatus of claim 3 in the secondary particle deflector comprises a magnetic deflector, a Wein filter, or an electrostatic deflector.

5. The apparatus of claim 1 in which the detector is positioned along the primary ion path and has a hole for passage of the primary ions.

6. The apparatus of claim 5 in which the detector comprises a conductive plate a scintillator, or a channel plate multiplier

7. The apparatus of claim 1 in which the target is maintained near ground electrical potential.

8. The apparatus of claim 1 in which the detector is maintained near ground electrical potential

9. The apparatus of claim 1 in which the detector is maintained at a potential that is positive with respect to the target to collect secondary electrons or negatively charged secondary ions or in which the secondary particle detector is maintained at a potential that is negative with respect to the target to collect secondary positive ions.

10. The apparatus of claim 1 in which the lens includes multiple lens elements, the lens element closest to the target being maintained at approximately the same electrical potential as the target.

11. The apparatus of claim 1 in which the lens includes multiple lens elements, the lens element farthest from the target being maintained at approximately the same electrical potential as the detector.

12. The apparatus of claim 1 further comprising a primary beam deflection electrode positioned between the secondary particle deflector and the lens.

13. A focused ion beam apparatus including a detector for detecting secondary particles, comprising:
an ion gun for generating primary ions and directing them toward a target;
a final lens for focusing the primary ions onto the target; and
a conductive plate for detecting secondary particles emitted from the target by the impact of the primary ions.

14. The apparatus of claim 13 in which the final lens defines an optical axis and in which the conductive plate is positioned essentially along the optical axis and has a hole for passing the ion beam.

15. The apparatus of claim 14 in which the conductive plate has a surface for the secondary particles to impact, the surface being substantially perpendicular to the optical axis.

16. The apparatus of claim 13 in which the secondary particles contacting the conductive plate cause a secondary particle electrical current and further comprising an amplifier for amplifying the secondary particle electrical current.

17. The apparatus of claim 13 in which the conductive plate is positioned between the target and the final lens.

18. The apparatus of claim 13 in which the conductive plate is electrically biased to collect secondary particles of a particular charge.

19. A method of collecting and detecting secondary particles emitted from a target in a focused ion beam column having a final ion lens and a primary ion path, comprising:
accelerating secondary particles away from the target and through the final ion lens; and
detecting the secondary particles that have traveled through the final lens.

20. The method of claim 19 in which accelerating the secondary particles away from the target includes providing an accelerating voltage on at least a portion of the final ion lens.

21. The method of claim 19 further comprising deflecting the secondary particles away from the primary ion path and towards a detector.

22. The method of claim 21 in which deflecting the secondary particles away from the primary ion path and towards a detector comprises deflecting the secondary particles using an electric field, a magnetic field, or a combination of an electric field and a magnetic field.

23. The method of claim 19 in which detecting the secondary particles includes detecting the secondary particles using a detector positioned along the primary ion path, the detector including a hole for passage of the primary ions.

24. The method of claim 19 in which the final lens includes multiple lens elements and in which accelerating the secondary particles away from the target includes maintaining the lens element closest to the target at the same electrical potential as the target and maintaining at least one of the other final lens elements at an electrical potential with respect to the target to accelerate the secondary particles away from the target.

25. The method of claim 24 in which maintaining the lens element closest to the target at the same electrical potential as the target includes maintaining the target at approximately ground potential.

26. The method of claim 24 in which detecting the secondary particles that have traveled through the final lens includes maintaining the detector at approximately ground potential.
